(19) United States
(12) Patent Application Publication
(10) Pub. No.: US 2022/0064805 A1
(43) Pub. Date: Mar. 3, 2022

(54) ELECTROCHEMICAL METHODS AND SYSTEMS FOR PRODUCING MONOSACCHARIDES

(71) Applicant: Cemvita Factory, Inc., Houston, TX (US)

(72) Inventor: Tahereh Karimi, Humble, TX (US)

(21) Appl. No.: 17/418,508

(22) PCT Filed: Dec. 6, 2019
(86) PCT No.: PCT/US19/64897

§ 371 Date: Jun. 25, 2021

Related U.S. Application Data
(60) Provisional application No. 62/786,403, filed on Dec. 29, 2018.

(51) Int. Cl. C25B 3/07; C25B 11/043; C25B 13/00; C25B 9/23; C25B 11/077; C25B 15/08

(52) U.S. Cl. CPC C25B 3/07; C25B 11/043; C25B 15/083; C25B 9/23; C25B 11/077; C25B 13/00

(57) ABSTRACT

The present disclosure is related to electrochemical methods of forming monosaccharides, and systems for generating the same. A benefit of the methods and systems disclosed herein can include the sustainable production of monosaccharides in an automated process. A benefit of the methods and systems herein can be the generation of monosaccharides from renewable source materials. An additional benefit of the methods and systems herein can include the use of abundant feedstocks, such as carbon dioxide, for the efficient generation of select monosaccharides for use as nutrients and for other useful applications. Another benefit of the methods and systems disclosed herein can include reduction of excess carbon dioxide from the environment.

### TECHNICAL FIELD

The present disclosure is related to electrochemical methods of forming monosaccharides, and systems for generating the same. A benefit of the methods and systems disclosed herein can include the sustainable production of monosaccharides in an automated process. A benefit of the methods and systems herein can be the generation of monosaccharides from renewable source materials. An additional benefit of the methods and systems herein can include the use of abundant feedstocks, such as carbon dioxide, for the efficient generation of select monosaccharides for use as nutrients and for other useful applications. Another benefit of the methods and systems disclosed herein can include reduction of excess carbon dioxide from the environment.

### BACKGROUND

Carbohydrates play a central role in providing energy for almost all biological processes. The formation of primary carbohydrates is believed to have arisen on Earth from abiotic and prebiotic reactions. Formose synthesis is a well-known type of prebiotic reaction, in which carbohydrate molecules are formed by condensation of formaldehyde molecules. Such reactions can potentially make use of molecular feedstocks as precursors for formaldehyde, including carbon dioxide as an abundant and economical carbon source. The production of biologically important carbohydrates, particularly monosaccharides such as glucose, can provide an important sustainable source of nutrients and other useful products. Glucose is a critical monosaccharide, because it serves as a substrate for the synthesis of almost all biomolecules, and has been used as a feedstock for a broad range of bio-manufacturing processes. Among the many useful applications can be the provision of safe and sustainable sources of nutrients for long-term space travel. Challenges remain, however, for the harnessing of sustainable feedstocks for the production of monosaccharides on an industrial scale.

The increased demand for power worldwide has led to an excess of carbon dioxide from burning fossil fuels such as oil and gas, contributing substantially to what many are calling a global warming crisis. Industry is so desperate to prevent carbon dioxide from entering the atmosphere that they have resorted to sequestering carbon dioxide from exhaust streams and the atmosphere. They then store the carbon dioxide in subterranean environments. However, all current known methods just remove carbon dioxide from the atmosphere by storing it under ground. They do not actually convert the carbon dioxide back into any other useful material.

There remains a need to produce monosaccharides through renewable and sustainable technologies. There remains a need to remove excess carbon dioxide from the atmosphere. There remains a need for methods to produce glucose and other monosaccharides from renewable feedstocks for use as nutrients and for other applications.

### SUMMARY

Embodiments herein are directed to methods of forming a monosaccharide. In such an embodiment, the method includes providing a formaldehyde source containing a formaldehyde aqueous electrolyte solution; feeding the formaldehyde source into a fluid flow path between and in contact with at least one pair of electrodes contained in a monosaccharide generating flow electrochemical cell, wherein the at least one pair of electrodes contains at least one monosaccharide generating catalyst; and forming an amount of the monosaccharide from the formaldehyde source by a condensation reaction in the presence of the at least one monosaccharide generating catalyst.

In certain embodiments of methods herein, the condensation reaction converts formaldehyde to the monosaccharide through the production of at least one intermediate species selected from the group consisting of glycolaldehyde, glyceraldehyde, and dihydroxyacetone. In various embodiments, the monosaccharide contains from 3 to 6 carbon atoms per molecule. In certain embodiments, the monosaccharide includes glucose, fructose, ribose, erythrose, or glyceraldehyde; or the monosaccharide includes glycerol.

In certain embodiments, the at least one monosaccharide generating catalyst contains sodium silicate, zinc-proline, or a combination thereof, and the monosaccharide is glucose; or the least one monosaccharide generating catalyst contains sodium borate, hydroxyapatite, or a combination thereof, and the monosaccharide is ribose. In certain embodiments, a total surface area of the at least one pair of electrodes and a total volume of the aqueous electrolyte solution have a surface area to volume ratio of from about 0.1 cm²/cm³ to about 1 cm²/cm³.

Various embodiments include performing the condensation reaction at a temperature of from about 100 degrees Celsius to about 300 degrees Celsius. Various embodiments include performing the condensation reaction at a pH of from about 7.5 to about 10.0.

In an embodiment, the method includes providing a methanol source containing a methanol aqueous electrolyte solution; feeding the methanol source into a fluid flow path between and in contact with at least one pair of electrodes contained in a formaldehyde generating flow electrochemical cell, wherein the at least one pair of electrodes contains at least one formaldehyde generating catalyst; and forming the formaldehyde source by converting the methanol source into formaldehyde and hydrogen gas in the presence of the at least one formaldehyde generating catalyst. In an embodiment, the at least one formaldehyde generating catalyst includes a cerium oxide catalyst.

In an embodiment, the method includes providing a carbon dioxide source; providing an electrochemical reactor cell comprising two fuel cell chambers separated by an ion exchange membrane, a positive electrode, a negative electrode, an hydrolysis aqueous electrolyte solution, and a power source; performing hydrolysis of water in the aqueous electrolyte solution in the electrochemical reactor cell to produce hydrogen gas; feeding the carbon dioxide source into the hydrolysis aqueous electrolyte solution and into contact with the negative electrode; and forming a methanol source by reacting the carbon dioxide source with the hydrogen gas in the presence of the negative electrode in a reduction reaction to produce methanol and oxygen gas.

In certain such embodiments, the method includes isolating, condensing, and pumping the methanol source into the fluid flow path of the formaldehyde generating flow electrochemical cell. In certain embodiments, the method includes isolating, condensing, and pumping the formaldehyde source into the fluid flow path of the monosaccharide generating flow electrochemical cell. In certain embodiments, the method includes recycling one or more of the water, the methanol, and the formaldehyde.

In certain embodiments, the power source includes solar power, sunlight, electrical power, or a combination thereof. In certain embodiments, the positive electrode contains copper, and the negative electrode contains zinc oxide.

In certain such embodiments, an efficiency of conversion of CO2 to monosaccharide is from about 40% to about 80% or greater, from about 60% to about 90% or greater, or about 80% to about 95%. In certain embodiments, a rate of formation of the monosaccharide is from about 40% to about 95%. In certain embodiments, a rate of conversion of formaldehyde to monosaccharide is from about 45% to about 99%.

In certain embodiments, the method includes feeding the carbon dioxide source into the hydrolysis aqueous electrolyte solution at a flow rate of from about 80 ml/min to about 110 ml/min. In certain embodiments, the method includes performing hydrolysis at a voltage from about 1.5 V to about 2.5 V. In certain embodiments, the method includes performing hydrolysis at a current density of about 15 mA or less. In certain embodiments, a concentration of carbon dioxide in the hydrolysis electrolyte solution is from about 0.5 g CO2/kg water to about 1.5 g CO2/kg water.

In an embodiment of methods of forming a monosaccharide herein, the method includes providing a carbon dioxide source containing a carbon dioxide aqueous electrolyte solution; feeding the carbon dioxide source into a fluid flow path between and in contact with at least one pair of electrodes contained in a formaldehyde generating flow electrochemical cell, wherein the at least one pair of electrodes contains at least one formaldehyde generating catalyst including a boron doped diamond catalyst; and forming a formaldehyde source by converting the carbon dioxide source into formaldehyde and hydrogen gas in the presence of the at least one formaldehyde generating catalyst.

In an embodiment, the method includes providing a carbon dioxide source containing a carbon dioxide aqueous electrolyte solution; feeding the carbon dioxide source into a fluid flow path between and in contact with at least one pair of electrodes contained in a formic acid generating flow electrochemical cell, wherein the at least one pair of electrodes contains at least one formic acid generating catalyst, thereby forming a formic acid source; and forming a formaldehyde source by converting the formic acid source into formaldehyde and water in the presence of at least one formaldehyde generating catalyst.

In an embodiment, the method includes providing a methane source containing a methane aqueous electrolyte solution; feeding the methane source into a fluid flow path between and in contact with at least one pair of electrodes contained in a methanol generating flow electrochemical cell, wherein the at least one pair of electrodes contains at least one methanol generating catalyst; and forming the methanol source by converting the methane source into methanol and hydrogen gas in the presence of the at least one methanol generating catalyst.

An embodiment of a method of forming a monosaccharide disclosed herein includes: providing a carbon dioxide source containing a carbon dioxide aqueous electrolyte solution; feeding the carbon dioxide source into a fluid flow path between and in contact with at least one pair of electrodes contained in a glycerol generating flow electrochemical cell, wherein the at least one pair of electrodes contains at least one glycerol generating catalyst; forming a glycerol source by converting the carbon dioxide source into glycerol in the presence of the at least one glycerol generating catalyst; feeding the glycerol source into a fluid flow path between and in contact with at least one pair of electrodes contained in a glyceraldehyde generating flow electrochemical cell, wherein the at least one pair of electrodes contains at least one glyceraldehyde generating catalyst; forming a glyceraldehyde source from the glycerol source in the presence of the at least one monosaccharide generating catalyst; feeding the glyceraldehyde source into a fluid flow path between and in contact with at least one pair of electrodes contained in a monosaccharide generating flow electrochemical cell, wherein the at least one pair of electrodes contains at least one monosaccharide generating catalyst; and forming an amount of the monosaccharide from the glyceraldehyde source by a condensation reaction in the presence of the at least one monosaccharide generating catalyst.

Embodiments herein are directed to an electrochemical system for generating a monosaccharide from carbon dioxide and water. Certain embodiments of such a system include: a hydrolysis electrochemical reactor cell comprising two fuel cell chambers separated by an ion exchange membrane, a positive electrode, a negative electrode, an hydrolysis aqueous electrolyte solution, and a power source; a carbon dioxide source connected to the hydrolysis aqueous electrolyte solution; a formaldehyde generator vessel containing a fluid flow path between and in contact with at least one pair of electrodes contained in a formaldehyde generating flow electrochemical cell, wherein the at least one pair of electrodes contains at least one formaldehyde generating catalyst; and a monosaccharide generator vessel containing a fluid flow path between and in contact with at least one pair of electrodes contained in a monosaccharide generating flow electrochemical cell, wherein the at least one pair of electrodes contains at least one monosaccharide generating catalyst.

In certain embodiments, the at least one monosaccharide generating catalyst contains sodium silicate, zinc-proline, or a combination thereof, and the monosaccharide is glucose. In certain embodiments, the least one monosaccharide generating catalyst contains sodium borate, hydroxyapatite, or a combination thereof, and the monosaccharide is ribose.

Certain embodiments of a system include a methanol isolation filter, a methanol condenser, and a pump connected to the hydrolysis electrochemical reactor cell and the formaldehyde generator vessel. Certain embodiments include a formaldehyde isolating filter and a formaldehyde pump connected to the formaldehyde generator vessel and the monosaccharide generator vessel. Certain embodiments include a monosaccharide isolation filter connected to the monosaccharide generator vessel. Certain embodiments include an oxygen receiver connected to the hydrolysis electrochemical reactor cell.

### BRIEF DESCRIPTION OF THE DRAWINGS

The foregoing summary, as well as the following detailed description of the embodiments, will be better understood when read in conjunction with the attached drawings. For the purpose of illustration, there are shown in the drawings some embodiments, which may be preferable. It should be understood that the embodiments depicted are not limited to the precise details shown. Unless otherwise noted, the drawings are not to scale.

FIG. 1 is a flow chart depicting an embodiment of a method of forming one or more monosaccharides herein.

FIG. 2 is a schematic illustration of a system for generating a monosaccharide according to embodiments herein.

FIG. 3 is a graph showing conversion of formaldehyde to monosaccharides over time according to embodiments herein.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one of glucose, fructose, ribose, erythrose, and glyceraldehyde” means one of glucose, fructose, ribose, erythrose, or glyceraldehyde; more than one of those objects in combination or independently; or any combination thereof

Unless otherwise noted, the term “about” refers to ±10% of the non-percentage number that is described, rounded to the nearest whole integer. For example, about 100 degrees Celsius, would include 90 to 110 degrees Celsius. Unless otherwise noted, the term “about” refers to ±5% of a percentage number. For example, about 40% would include 35 to 45%. When the term “about” is discussed in terms of a range, then the term refers to the appropriate amount less than the lower limit and more than the upper limit. For example, from about 100 degrees Celsius to about 300 degrees Celsius would include from 90 to 330 degrees Celsius.

Unless otherwise noted, properties (height, width, length, ratio etc.) as described herein are understood to be averaged measurements.

Unless otherwise noted, the terms “provide”, “provided” or “providing” refer to the supply, production, purchase, manufacture, assembly, formation, selection, configuration, conversion, introduction, addition, or incorporation of any element, amount, component, reagent, quantity, measurement, or analysis of any method or system of any embodiment herein.

Unless otherwise noted, the term “monosaccharide” refers to a monosaccharide containing from 3 to 6 carbon atoms per molecule, including but not limited to glucose, fructose, ribose, erythrose, glyceraldehyde, and combinations thereof. Unless otherwise noted, the term “monosaccharide” herein also refers to glycerol.

For ease of copying, traditional formulas such as CO₂ are often written as CO2, without using the subscript for the number. Unless otherwise noted CO₂ and CO2 are interchangeable. Similarly, CeO₂ and CeO2 are interchangeable.

Unless otherwise noted, a “fluid flow path” refers to a path of fluid flow that can be can be controlled and made discontinuous by the opening and closing of valves, and the like, so long as the fluid flow path is capable of forming a continuous, connected path of fluid flow.

Mechanisms of formation of carbohydrates have been the subject of much interest, because of their importance for a greater understanding of the origins of life on Earth. Almost all biological processes require carbohydrates in order to function. The primary monosaccharides are believed to have originated from abiotic and prebiotic reactions, most notably the prebiotic formose synthesis of monosaccharide molecules from condensation of formaldehyde. Among the biologically important monosaccharides, glucose is of key importance for its central role in providing energy for essential biological functions. Monosaccharides such as glucose have innumerable beneficial uses in various industries and research efforts, as well as representing a key source of nutrients. The generation of monosaccharides on an industrial scale could provide a benefit of a safe food source for humans, including an important sustainable food source during long-term space missions.

One of the major constraints of long-duration space travel, such as landing people on Mars, is the production of a safe, nutritious food system. State-of-the-art (SOA) technologies for NASA applications either do not exist or are too small in scale. CO2 conversion systems that do exist are heavy, inefficient, or consume too much power. Other approaches for production of biomass rely on bacteria and need a lot of care. Embodiments of the present disclosure can provide the benefits of a system that is self-regenerative and requires very little tending, and a bio-regenerative food system with creation of base nutrients like monosaccharides on a large scale. If nutrient biomass is made real-time, packaging and shelf life are no longer issues. Additionally, embodiments of the present disclosure can provide a benefit of an ability to be configured to make IV fluids for both nutrient and pharmaceutics delivery for crew health care.

Chemical reactions that produce monosaccharides can potentially make use of molecular feedstocks as precursors for formaldehyde or other monosaccharide building blocks. One such feedstock is carbon dioxide, which provides an abundant, renewable, and economical carbon source.

Reduction of atmospheric carbon dioxide levels is a key to mitigating or reversing climate change. Although carbon capture methods such as Carbon Capture and Storage (CCS) can present cost effective and affordable ways to reduce carbon dioxide emissions, the problem remains that the carbon dioxide is merely being stored underground until it escapes. Therefore, currently available methods do not provide a sustainable solution to reduce excess carbon dioxide in the atmosphere. There remains a need to remove excess carbon dioxide from the atmosphere in sustainable ways. There remains a need for technologies that can harness the over-abundance of carbon dioxide to make useful products, and for other applications that are beneficial to industry and the environment.

Embodiments of the present disclosure can provide a benefit of methods and systems for the production of biologically important monosaccharides using carbon dioxide, water, and light energy as feedstocks. Such embodiments can provide an important benefit of a sustainable and renewable source of nutrients and other useful products, while serving to help remove excess carbon dioxide from the atmosphere.

Challenges remain for the harnessing of sustainable feedstocks such as carbon dioxide for the production of monosaccharides on an industrial scale. Electrochemical reactions can be utilized to carry out condensation reactions of carbon dioxide and other precursors for the production of monosaccharide molecules. However, gas-liquid mass transfer is a major challenge during the conversion of CO2 to monosaccharides in condensation reactions, having a substantial effect on the efficiency of the reactions. Additional factors can affect gas-liquid transfer, including electrode surface area, electrolyte volume, pH, temperature, and CO2 concentration in the electrolyte fluid. Another challenge is the specificity of the formose reaction to produce particular monosaccharides with high purity. Embodiments of the present disclosure can provide a benefit of electrochemical methods and systems for the efficient and specific formation of a monosaccharide from carbon dioxide, as well as other precursors.

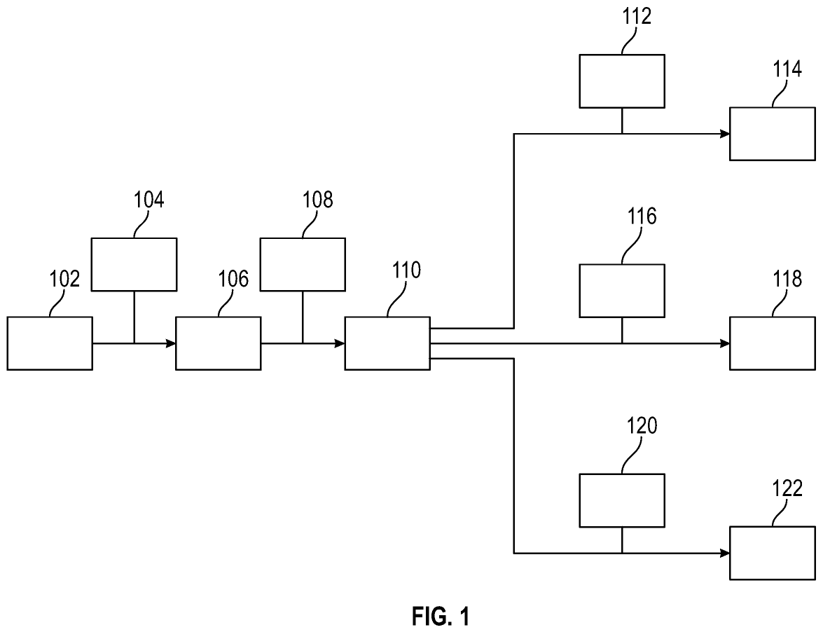

The present disclosure relates to electrochemical methods of forming a monosaccharide, and systems therefor. As a general overview of a method disclosed herein, referring to FIG. 1, embodiments of the method includes providing a carbon dioxide source 102; reacting the carbon dioxide source with hydrogen gas in the presence of Cu/ZnO electrode 104 to form a methanol source 106; converting the methanol source in the presence of CeO2 catalyst 108 to form formaldehyde source 110; converting the formaldehyde source by self-condensation reaction 112 into glycerol 114; converting the formaldehyde source in the presence of Na borate/Hydroxy apatite catalyst 116 to form ribose 118; or converting the formaldehyde source in the presence of Na silicate catalyst 120 into glucose 122.

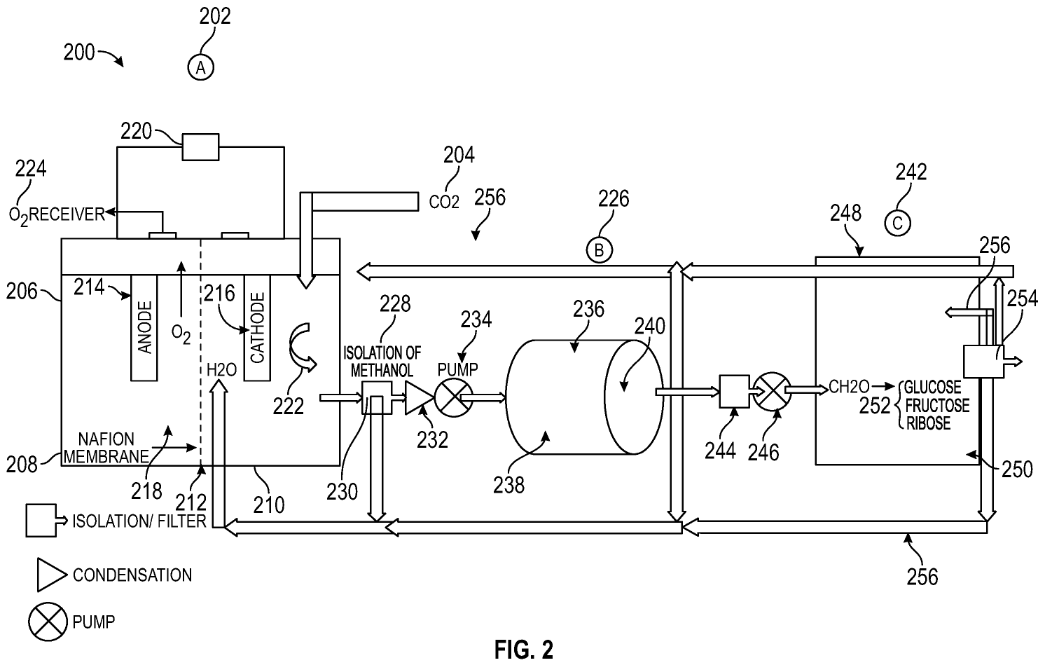

As an illustration of a system disclosed herein, referring to FIG. 2, electrochemical system 200 includes water electrolysis, CO2 reduction and methanol formation system 202, formaldehyde formation system 226 and formaldehyde conversion system 242. Water electrolysis, CO2 reduction and methanol formation system 202 includes CO2 source 204, electrochemical reactor cell 206 having fuel cell chambers 208 and 210 separated by ion exchange membrane 212; positive electrode 214, negative electrode 216, hydrolysis aqueous electrolyte solution 218, power source 220, produced hydrogen gas 222, and oxygen receiver 224. Formaldehyde formation system 226 includes methanol source 228, methanol isolation filter 230, methanol condenser 232, methanol pump 234, formaldehyde generator 236, CeO2 formaldehyde generating catalyst 238, and formaldehyde source 240. Formaldehyde conversion system 242 includes formaldehyde isolation filter 244, formaldehyde pump 246, monosaccharide generator 248, monosaccharide generating catalyst 250, formed monosaccharide 252, and monosaccharide isolation filter 254. Electrochemical system 200 includes chemical intermediate and by-product recycling pipelines 256.

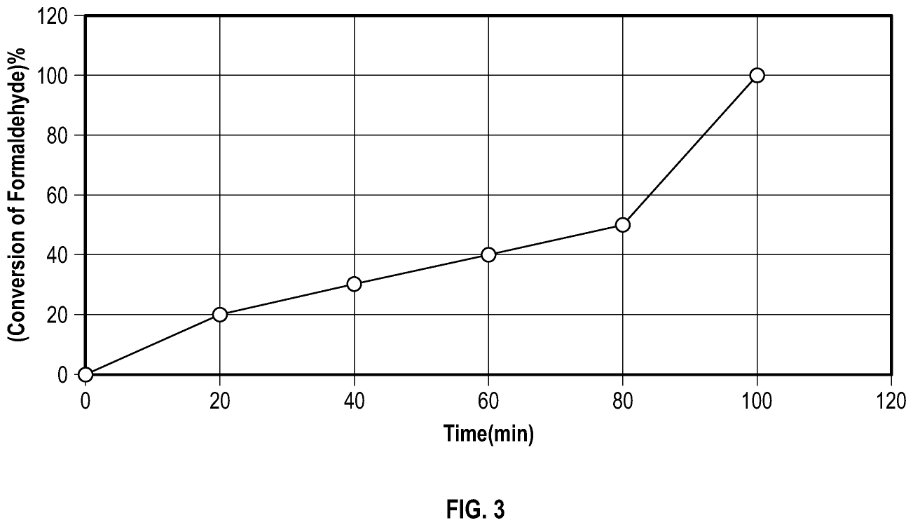

FIG. 3 is a graph showing conversion of formaldehyde to monosaccharides over time according to embodiments herein.

### Embodiments of Methods of Forming a Monosaccharide

Embodiments herein are directed to methods of forming a monosaccharide. In such an embodiment, the method includes providing a formaldehyde source containing a formaldehyde aqueous electrolyte solution; feeding the formaldehyde source into a fluid flow path between and in contact with at least one pair of electrodes contained in a monosaccharide generating flow electrochemical cell, wherein the at least one pair of electrodes contains at least one monosaccharide generating catalyst; and forming an amount of the monosaccharide from the formaldehyde source by a condensation reaction in the presence of the at least one monosaccharide generating catalyst.

In certain embodiments of methods herein, the condensation reaction converts formaldehyde to the monosaccharide through the production of at least one intermediate species selected from the group consisting of glycolaldehyde, glyceraldehyde, and dihydroxyacetone. In various embodiments, the monosaccharide contains from 3 to 6 carbon atoms per molecule. In certain embodiments, the monosaccharide includes a triose, a tetrose, a pentose, a hexose, or a combination thereof. In certain embodiments, the monosaccharide includes glucose, fructose, ribose, erythrose, or glyceraldehyde; or the monosaccharide includes glycerol. In certain embodiments, the monosaccharide can include erythrose, threose, arabinose, xylose, lyxose, allose, altrose, mannose, gulose, idose, galactose, talose, or combinations thereof. In certain embodiments, the method converts formaldehyde into one or more monosaccharides.

In certain embodiments, the at least one monosaccharide generating catalyst contains sodium silicate, zinc-proline, or a combination thereof, and the monosaccharide is glucose. In certain embodiments, the least one monosaccharide generating catalyst contains sodium borate, hydroxyapatite, or a combination thereof, and the monosaccharide is ribose. Such embodiments can provide a benefit of selecting one or more monosaccharide generating catalysts for specificity of the formose reaction to produce one or more selected monosaccharides. In certain embodiments, a total surface area of the at least one pair of electrodes and a total volume of the aqueous electrolyte solution have a surface area to volume ratio of from about 0.1 cm²/cm³ to about 1 cm²/cm³. Such embodiments can provide a benefit of improving efficiency of the formose reactions.

Various embodiments include performing the condensation reaction at a temperature of from about 100 degrees Celsius to about 300 degrees Celsius. Certain embodiments include performing the condensation reaction at a temperature of from about 125 degrees Celsius to about 275 degrees Celsius. Certain embodiments include performing the condensation reaction at a temperature of from about 150 degrees Celsius to about 250 degrees Celsius. Various embodiments include performing the condensation reaction at a pH of from about 7.5 to about 10.0. Certain embodiments include performing the condensation reaction at a pH of from about 8.0 to about 9.5. Certain embodiments include performing the condensation reaction at a pH of from about 8.5 to about 9.0. Such embodiments can provide a benefit of improving efficiency of the formose reactions.

In an embodiment, the method includes providing a methanol source containing a methanol aqueous electrolyte solution; feeding the methanol source into a fluid flow path between and in contact with at least one pair of electrodes contained in a formaldehyde generating flow electrochemical cell, wherein the at least one pair of electrodes contains at least one formaldehyde generating catalyst; and forming the formaldehyde source by converting the methanol source into formaldehyde and hydrogen gas in the presence of the at least one formaldehyde generating catalyst. In an embodiment, the at least one formaldehyde generating catalyst includes a cerium oxide catalyst.

In an embodiment, the method includes providing a carbon dioxide source; providing an electrochemical reactor cell comprising two fuel cell chambers separated by an ion exchange membrane, a positive electrode, a negative electrode, an hydrolysis aqueous electrolyte solution, and a power source; performing hydrolysis of water in the aqueous electrolyte solution in the electrochemical reactor cell to produce hydrogen gas; feeding the carbon dioxide source into the hydrolysis aqueous electrolyte solution and into contact with the negative electrode; and forming a methanol source by reacting the carbon dioxide source with the hydrogen gas in the presence of the negative electrode in a reduction reaction to produce methanol and oxygen gas. In certain embodiments, the carbon dioxide source can be derived from ambient gas aboard a space-borne vehicle, an orbital platform, or an orbital vehicle. In certain embodiments, the carbon dioxide source can be derived from atmospheric carbon dioxide, carbon dioxide that has been captured from refining and other industrial processes, or carbon dioxide stored in a subterranean formation or storage tank.

In certain such embodiments, the method includes isolating, condensing, and pumping the methanol source into the fluid flow path of the formaldehyde generating flow electrochemical cell. In certain embodiments, the method includes isolating, condensing, and pumping the formaldehyde source into the fluid flow path of the monosaccharide generating flow electrochemical cell. In certain embodiments, the method includes recycling one or more of the water, the methanol, and the formaldehyde. Such embodiments can provide a benefit of a sustainable, continuous production of monosaccharides in an automated process.

In certain embodiments, the power source includes solar power, sunlight, electrical power, or a combination thereof. In certain embodiments, the positive electrode contains copper, and the negative electrode contains zinc oxide.

In certain such embodiments, an efficiency of conversion of CO2 to monosaccharide is from about 40% to about 80% or greater, from about 60% to about 90% or greater, or about 80% to about 95%. In certain embodiments, a rate of formation of the monosaccharide is from about 40% to about 95%. In certain embodiments, a rate of conversion of formaldehyde to monosaccharide is from about 45% to about 99%.

In certain embodiments, the method includes feeding the carbon dioxide source into the hydrolysis aqueous electrolyte solution at a flow rate of from about 80 ml/min to about 110 ml/min. Certain embodiments include feeding the carbon dioxide source into the hydrolysis aqueous electrolyte solution at a flow rate of from about 85 ml/min to about 105 ml/min. Certain embodiments include feeding the carbon dioxide source into the hydrolysis aqueous electrolyte solution at a flow rate of from about 90 ml/min to about 100 ml/min. In certain embodiments, the method includes performing hydrolysis at a voltage from about 1.5 V to about 2.5 V. Certain embodiments include performing hydrolysis at a voltage from about 1.9 V to about 2.1 V. In certain embodiments, the method includes performing hydrolysis at a current density of about 15 mA or less. Certain embodiments include performing hydrolysis at a current density of about 10 mA or less. Certain embodiments include performing hydrolysis at a current density of about 5 mA or less. In certain embodiments, a concentration of carbon dioxide in the hydrolysis electrolyte solution is from about 0.5 g CO2/kg water to about 1.5 g CO₂/kg water.

In an embodiment of methods of forming a monosaccharide herein, the method includes providing a carbon dioxide source containing a carbon dioxide aqueous electrolyte solution; feeding the carbon dioxide source into a fluid flow path between and in contact with at least one pair of electrodes contained in a formaldehyde generating flow electrochemical cell, wherein the at least one pair of electrodes contains at least one formaldehyde generating catalyst including a boron doped diamond catalyst; and forming a formaldehyde source by converting the carbon dioxide source into formaldehyde and hydrogen gas in the presence of the at least one formaldehyde generating catalyst.

In an embodiment, the method includes providing a carbon dioxide source containing a carbon dioxide aqueous electrolyte solution; feeding the carbon dioxide source into a fluid flow path between and in contact with at least one pair of electrodes contained in a formic acid generating flow electrochemical cell, wherein the at least one pair of electrodes contains at least one formic acid generating catalyst, thereby forming a formic acid source; and forming a formaldehyde source by converting the formic acid source into formaldehyde and water in the presence of at least one formaldehyde generating catalyst.

In an embodiment, the method includes providing a methane source containing a methane aqueous electrolyte solution; feeding the methane source into a fluid flow path between and in contact with at least one pair of electrodes contained in a methanol generating flow electrochemical cell, wherein the at least one pair of electrodes contains at least one methanol generating catalyst; and forming the methanol source by converting the methane source into methanol and hydrogen gas in the presence of the at least one methanol generating catalyst.

In an embodiment of the method, one or more steps of the method are performed in a closed system, wherein the closed system is continuous and integrated. In an embodiment, a majority of steps, including each step, of the methods disclosed herein are performed within a closed system. In an embodiment, water electrolysis step, formaldehyde formation step, or formaldehyde step, or any combination thereof are a closed system. A closed system is one that is sealed, including hermetically sealed, off from the ambient environment. One benefit of performing the methods disclosed herein in a closed system can include making the methods suitable for use in low gravity environments and/or in the confined and highly controlled environments of space travel.

An embodiment of a method of forming a monosaccharide disclosed herein includes: providing a carbon dioxide source containing a carbon dioxide aqueous electrolyte solution; feeding the carbon dioxide source into a fluid flow path between and in contact with at least one pair of electrodes contained in a glycerol generating flow electrochemical cell, wherein the at least one pair of electrodes contains at least one glycerol generating catalyst; forming a glycerol source by converting the carbon dioxide source into glycerol in the presence of the at least one glycerol generating catalyst; feeding the glycerol source into a fluid flow path between and in contact with at least one pair of electrodes contained in a glyceraldehyde generating flow electrochemical cell, wherein the at least one pair of electrodes contains at least one glyceraldehyde generating catalyst; forming a glyceraldehyde source from the glycerol source in the presence of the at least one monosaccharide generating catalyst; feeding the glyceraldehyde source into a fluid flow path between and in contact with at least one pair of electrodes contained in a monosaccharide generating flow electrochemical cell, wherein the at least one pair of electrodes contains at least one monosaccharide generating catalyst; and forming an amount of the monosaccharide from the glyceraldehyde source by a condensation reaction in the presence of the at least one monosaccharide generating catalyst.

### Embodiments of Systems for Generating a Monosaccharide

Embodiments herein are directed to an electrochemical system for generating a monosaccharide from carbon dioxide and water. Certain embodiments of such a system include: a hydrolysis electrochemical reactor cell comprising two fuel cell chambers separated by an ion exchange membrane, a positive electrode, a negative electrode, an hydrolysis aqueous electrolyte solution, and a power source; a carbon dioxide source connected to the hydrolysis aqueous electrolyte solution; a formaldehyde generator vessel containing a fluid flow path between and in contact with at least one pair of electrodes contained in a formaldehyde generating flow electrochemical cell, wherein the at least one pair of electrodes contains at least one formaldehyde generating catalyst; and a monosaccharide generator vessel containing a fluid flow path between and in contact with at least one pair of electrodes contained in a monosaccharide generating flow electrochemical cell, wherein the at least one pair of electrodes contains at least one monosaccharide generating catalyst.

In certain embodiments, the carbon dioxide source can be derived from ambient gas aboard a space-borne vehicle, an orbital platform, or an orbital vehicle. In certain embodiments, the carbon dioxide source can be derived from atmospheric carbon dioxide, carbon dioxide that has been captured from refining and other industrial processes, or carbon dioxide stored in a subterranean formation or storage tank. In certain embodiments, the power source includes solar power, sunlight, electrical power, or a combination thereof. In certain embodiments, the positive electrode contains copper, and the negative electrode contains zinc oxide.

In certain embodiments, the at least one monosaccharide generating catalyst contains sodium silicate, zinc-proline, or a combination thereof, and the monosaccharide is glucose. In certain embodiments, the least one monosaccharide generating catalyst contains sodium borate, hydroxyapatite, or a combination thereof, and the monosaccharide is ribose. Such embodiments can provide a benefit of one or more monosaccharide generating catalysts for specificity of the formose reaction for generation of one or more selected monosaccharides.

Certain embodiments of a system include a methanol isolation filter, a methanol condenser, and a pump connected to the hydrolysis electrochemical reactor cell and the formaldehyde generator vessel. Certain embodiments include a formaldehyde isolating filter and a formaldehyde pump connected to the formaldehyde generator vessel and the monosaccharide generator vessel. Certain embodiments include a monosaccharide isolation filter connected to the monosaccharide generator vessel. Certain embodiments include an oxygen receiver connected to the hydrolysis electrochemical reactor cell. In certain embodiments, the system includes one or more pipelines for recycling one or more of the water, the methanol, and the formaldehyde. Such embodiments can provide a benefit of a system for sustainable, continuous, automated production of monosaccharides.

In an embodiment of the system, one or more parts of the system are performed in a closed system, wherein the closed system is continuous and integrated. In an embodiment, a majority of parts of the system, including each part, of the systems disclosed herein are part of a closed system. In an embodiment, water electrolysis system, formaldehyde formation system, or formaldehyde conversion, or any combination thereof are a closed system. A closed system is one that is sealed, including hermetically sealed, off from the ambient environment. One benefit of performing the methods disclosed herein in a closed system can include making the system suitable for use in low gravity environments and/or in the confined and highly controlled environments of space travel.

### EXAMPLES

### Example I: Conversion of CO2 and Water to Methanol

The process starts with the conversion of CO2 to methanol in an electrochemical reactor (see FIG. 2). The electrochemical reactor is made of two chambers which are separated from each other by Nafion®-117 ion-exchange membrane (Nafion, US). The fuel-cell chambers were purchased from Adams & Chittenden Scientific Glass, Inc (USA). A pair of Cu/ZnO electrodes were used to operate the CO2 reduction reaction. Hydrolysis of water was performed at voltage 1.9 V. The hydrogen (released from electrolysis of water) was used for CO2 reduction at the cathode. Pure CO2 was then bubbled into the system with the flow rate of 100 ml min-1. The CO2 reduction reaction was done at the cathode. Reduction of CO2 in the presence of Cu/ZnO leads to the production of methanol.

A continuous flow cell chemistry reactor system (Flow Syn™, Uniqsis, UK) is be used to increase the liquid-gas mass transfer. The system can carry out reactions up to 300° C. and 200 bars. The required energy for the electrochemical reactions is be obtained from sunlight. Applying this continuous flow chemistry system, the products of each stage can be transferred to the next unit by using a pump. After each reaction, all intermediates and byproducts are isolated and recycled (see FIG. 2). The output product of each stage will be isolated and analyzed using a high-pressure liquid chromatography (HPLC) system (Ataka pure and fraction collection-F9C for isolation and analytical assays different sugars).

### Example II: Conversion of Methanol to Formaldehyde

Large scale production of formaldehyde from CO2 was performed by applying currently available methods of electrochemical engineering. CeO2 was applied, as a catalyst, to convert methanol to formaldehyde. The reaction was done at 60° C., and using concentrated methanol (96%). Formaldehyde measurement was done by applying a fluorometric formaldehyde assay kit (Abacam).

### Example III: Conversion of Formaldehyde to Biologically Important Monosaccharides

In order to convert formaldehyde to glucose, the formose reaction was applied. The formose reaction was done in an alkaline environment at high temperature (100° C.). The formose reaction was done in the presence of sodium silicate (as a catalyst) to produce glucose. Other catalysts including zinc and proline, also will be used to produce glucose. Hydroxyapatite and sodium borate will be used to produce ribose. Glycerol will be produced by a self-condensation reaction of formaldehyde molecules at 100° C. and pH 8 (see FIG. 1).

After the operation of each reaction, samples were collected from the reactions after 2 hours. Samples were stored at −20° C. until the analysis assays. Glucose measurement assays were done applying Megazyme-Glucose measurement assays. HPLC (high performance liquid chromatography) will be applied to measure the concentration of all different sugars and glycerol after the formose reactions.

### Example IV: Features of the Flow Cell Chemistry Reactor System

The flow cell reactor system is made of two compartments including,
1—A flow fuel cell (with the final volume of 500 ml) where the water hydrolysis and CO2 reduction reaction happens.
2—Two stainless steel chambers where chemical reactions of formaldehyde and monosaccharide synthesis happen (each one 250 mL).

Features of the flow chemistry reactor system:
Carry out reactions up to 300° C., 200 bar and 100 ml/min.
A wide range of coil, chip and column reactor modules
Latest generation chemically inert high-pressure pump heads
Durable coated stainless-steel casework

The following materials were applied as catalysts:

Cu, ZnO, CeO2, Sodium Borate, Sodium Silicate

The total size of the system: 50×50×40 Cm

Total weight of the system: less than 4 Kg

### Example V: Efficiency of Monosaccharide Formation

In order to improve the efficiency of reactions, and to optimize the gas-liquid mass transfer, firstly the gas-liquid mass transfer model was used in the steady state. The mass balance for CO2 dissociation into the electrolyte and its consumption at the cathode were defined by the following formula.

N_{CO2} Sc V⁻¹=K_{L}a(C*−C^{b})

Where N_{CO2} is the flux of CO2 consumed at the electrode surface, S_{c} is the surface area of the catalyst, V is the electrolyte volume, and KL is the liquid mass transfer coefficient. A is the interfacial surface area bubbles per volume of liquid, C* is the solubility limit of CO2 in water and C^{b} is the concentration of CO2 in the electrolyte.

Based on a preliminary analysis, an increase in the consumption of CO2 at the cathode will increase the mass transfer coefficient increases. Therefore, the electrolyte saturation was maintained by an increase in the magnitude of the mass transfer coefficient. To this end, a flow cell system was designed to enhance the gas-liquid mass transfer.

According to a preliminary analysis, the system can generate at least 12.6 grams of sugar by consuming 26.4 grams of liquid CO2 over 24 hours. Also, results indicated that current densities up to 15 mA are sufficient for CO2 reduction reaction in the presence of Cu. Water hydrolysis was done at 1.9 V.

The percentage of formaldehyde conversion to monosaccharides by time was measured (see FIG. 3). Different catalysts will be adapted for the selective production of different monosaccharides.

According to the gas-liquid mass transfer model, it can be concluded that pH, temperature, and the ratio of catalyst surface area to the electrolyte volume are critical to the gas-liquid mass transfer, and consequently the CO2 conversion rate. Therefore, the efficiency of CO2 conversion was increased by an increase in the ratio of SN (where S is the electrocatalyst surface area and V is the volume of electrolyte). Also, the temperature and pH of the electrolyte were monitored constantly to provide a consistent CO2 concentration.

What is claimed is:

1. A method of forming a monosaccharide comprising:
providing a formaldehyde source containing a formaldehyde aqueous electrolyte solution;
feeding the formaldehyde source into a fluid flow path between and in contact with at least one pair of electrodes contained in a monosaccharide generating flow electrochemical cell, wherein the at least one pair of electrodes contains at least one monosaccharide generating catalyst; and
forming an amount of the monosaccharide from the formaldehyde source by a condensation reaction in the presence of the at least one monosaccharide generating catalyst.

2. The method of claim 1, further comprising:
providing a methanol source containing a methanol aqueous electrolyte solution;
feeding the methanol source into a fluid flow path between and in contact with at least one pair of electrodes contained in a formaldehyde generating flow electrochemical cell, wherein the at least one pair of electrodes contains at least one formaldehyde generating catalyst; and
forming the formaldehyde source by converting the methanol source into formaldehyde and hydrogen gas in the presence of the at least one formaldehyde generating catalyst.

3. The method of claim 2, further comprising:
providing a carbon dioxide source;
providing an electrochemical reactor cell comprising two fuel cell chambers separated by an ion exchange membrane, a positive electrode, a negative electrode, an hydrolysis aqueous electrolyte solution, and a power source;
performing hydrolysis of water in the aqueous electrolyte solution in the electrochemical reactor cell to produce hydrogen gas;
feeding the carbon dioxide source into the hydrolysis aqueous electrolyte solution and into contact with the negative electrode; and
forming the methanol source by reacting the carbon dioxide source with the hydrogen gas in the presence of the negative electrode in a reduction reaction to produce methanol and oxygen gas.

4. The method of claim 1, further comprising:
providing a carbon dioxide source containing a carbon dioxide aqueous electrolyte solution;
feeding the carbon dioxide source into a fluid flow path between and in contact with at least one pair of electrodes contained in a formaldehyde generating flow electrochemical cell, wherein the at least one pair of electrodes contains at least one formaldehyde generating catalyst including a boron doped diamond catalyst; and
forming the formaldehyde source by converting the carbon dioxide source into formaldehyde and hydrogen gas in the presence of the at least one formaldehyde generating catalyst.

5. The method of claim 1, further comprising:
providing a carbon dioxide source containing a carbon dioxide aqueous electrolyte solution;
feeding the carbon dioxide source into a fluid flow path between and in contact with at least one pair of electrodes contained in a formic acid generating flow electrochemical cell, wherein the at least one pair of electrodes contains at least one formic acid generating catalyst, thereby forming a formic acid source; and
forming the formaldehyde source by converting the formic acid source into formaldehyde and water in the presence of at least one formaldehyde generating catalyst.

6. The method of claim 2, further comprising:
providing a methane source containing a methane aqueous electrolyte solution;
feeding the methane source into a fluid flow path between and in contact with at least one pair of electrodes contained in a methanol generating flow electrochemical cell, wherein the at least one pair of electrodes contains at least one methanol generating catalyst; and
forming the methanol source by converting the methane source into methanol and hydrogen gas in the presence of the at least one methanol generating catalyst.

7. A method of forming a monosaccharide comprising:
providing a carbon dioxide source containing a carbon dioxide aqueous electrolyte solution;
feeding the carbon dioxide source into a fluid flow path between and in contact with at least one pair of electrodes contained in a glycerol generating flow electrochemical cell, wherein the at least one pair of electrodes contains at least one glycerol generating catalyst;
forming a glycerol source by converting the carbon dioxide source into glycerol in the presence of the at least one glycerol generating catalyst;
feeding the glycerol source into a fluid flow path between and in contact with at least one pair of electrodes contained in a glyceraldehyde generating flow electrochemical cell, wherein the at least one pair of electrodes contains at least one glyceraldehyde generating catalyst;
forming a glyceraldehyde source from the glycerol source in the presence of the at least one monosaccharide generating catalyst;
feeding the glyceraldehyde source into a fluid flow path between and in contact with at least one pair of electrodes contained in a monosaccharide generating flow electrochemical cell, wherein the at least one pair of electrodes contains at least one monosaccharide generating catalyst; and
forming an amount of the monosaccharide from the glyceraldehyde source by a condensation reaction in the presence of the at least one monosaccharide generating catalyst.

8. The method of claim 1, wherein the condensation reaction converts formaldehyde to the monosaccharide through the production of at least one intermediate species selected from the group consisting of glycolaldehyde, glyceraldehyde, and dihydroxyacetone.

9. The method of claim 1, wherein the monosaccharide contains from 3 to 6 carbon atoms per molecule.

10. The method of claim 1, wherein the monosaccharide is selected from the group consisting of glucose, fructose, ribose, erythrose, and glyceraldehyde; or the monosaccharide includes glycerol.

11. The method of claim 1, wherein the at least one monosaccharide generating catalyst contains sodium silicate, zinc-proline, or a combination thereof, and the monosaccharide is glucose; or the least one monosaccharide generating catalyst contains sodium borate, hydroxyapatite, or a combination thereof, and the monosaccharide is ribose.

12. The method of claim 1, wherein a total surface area of the at least one pair of electrodes and a total volume of the aqueous electrolyte solution have a surface area to volume ratio of from about 0.1 cm²/cm³ to about 1 cm²/cm³.

13. The method of claim 1, further comprising performing the condensation reaction at a temperature of from about 100 degrees Celsius to about 300 degrees Celsius, or performing the condensation reaction at a pH of from about 7.5 to about 10.0.

14. The method of claim 3, wherein an efficiency of conversion of CO2 to monosaccharide is from about 40% to about 80% or greater, from about 60% to about 90% or greater, or about 80% to about 95%; or a rate of formation of the monosaccharide is from about 40% to about 95%; or a rate of conversion of formaldehyde to monosaccharide is from about 45% to about 99%.

15. The method of claim 3, comprising feeding the carbon dioxide source into the hydrolysis aqueous electrolyte solution at a flow rate of from about 80 ml/min to about 110 ml/min, or performing hydrolysis at a voltage from about 1.5 V to about 2.5 V, or performing hydrolysis at a current density of about 15 mA or less, or wherein a concentration of carbon dioxide in the hydrolysis electrolyte solution is from about 0.5 g CO2/kg water to about 1.5 g CO₂/kg water.

16. The method of claim 3, wherein the power source includes solar power, sunlight, electrical power, or a combination thereof

17. The method of claim 3, wherein the positive electrode contains copper, and the negative electrode contains zinc oxide.

18. The method of claim 2, wherein the at least one formaldehyde generating catalyst includes a cerium oxide catalyst.

19. The method of claim 3, further comprising isolating, condensing, and pumping the methanol source into the fluid flow path of the formaldehyde generating flow electrochemical cell; or isolating, condensing, and pumping the formaldehyde source into the fluid flow path of the monosaccharide generating flow electrochemical cell.

20. The method of claim 3, further comprising recycling one or more of the water, the methanol, and the formaldehyde.

21. An electrochemical system for generating a monosaccharide from carbon dioxide and water comprising:
a hydrolysis electrochemical reactor cell comprising two fuel cell chambers separated by an ion exchange membrane, a positive electrode, a negative electrode, an hydrolysis aqueous electrolyte solution, and a power source;
a carbon dioxide source connected to the hydrolysis aqueous electrolyte solution;
a formaldehyde generator vessel containing a fluid flow path between and in contact with at least one pair of electrodes contained in a formaldehyde generating flow electrochemical cell, wherein the at least one pair of electrodes contains at least one formaldehyde generating catalyst; and
a monosaccharide generator vessel containing a fluid flow path between and in contact with at least one pair of electrodes contained in a monosaccharide generating flow electrochemical cell, wherein the at least one pair of electrodes contains at least one monosaccharide generating catalyst.

22. The system of claim 21, further comprising a methanol isolation filter, a methanol condenser, and a pump connected to the hydrolysis electrochemical reactor cell and the formaldehyde generator vessel.

23. The system of claim 21, further comprising a formaldehyde isolating filter and a formaldehyde pump connected to the formaldehyde generator vessel and the monosaccharide generator vessel.

24. The system of claim 21, further comprising a monosaccharide isolation filter connected to the monosaccharide generator vessel.

25. The system of claim 21, wherein the at least one monosaccharide generating catalyst contains sodium silicate, zinc-proline, or a combination thereof, and the monosaccharide is glucose; or the least one monosaccharide generating catalyst contains sodium borate, hydroxyapatite, or a combination thereof, and the monosaccharide is ribose.

26. The system of claim 21, further comprising an oxygen receiver connected to the hydrolysis electrochemical reactor cell.
